(19) United States
(12) Patent Application Publication
(10) Pub. No.: US 2013/0323824 A1
(43) Pub. Date: Dec. 5, 2013

(54) CONTROL DEVICE FOR A FOOD WASTE DISPOSAL APPARATUS

(71) Applicant: CHUN IL KOH, Seoul (KR)

(72) Inventor: CHUN IL KOH, Seoul (KR)

(21) Appl. No.: 13/487,213

(22) Filed: Jun. 3, 2012

(51) Int. Cl. C12M 1/36; C12M 1/00

(52) U.S. Cl. 435/286.5; 435/290.1; 435/286.1

(57) ABSTRACT

This invention relates to a control device for a food waste disposal apparatus. The control device is comprised of a touch screen, a memory device to perform a specific function to control the food waste disposal apparatus. The touch screen of the control device displays a plurality of input button images so that a user can click them by fingers to prosecute. The main menu of the touch screen is comprised of auto working mode, total monitoring mode, manual working mode, time setting mode, alarm message mode and function setting mode. The sub-menu of function setting mode further includes cumulative weight setting and part replacement cycle setting.

### BACKGROUND OF THE INVENTION

The present invention relates to a control device for use in controlling a food waste disposal apparatus. More particularly, this invention relates to a control device for a food waste disposal apparatus comprising auto working mode, manual working mode, alarm message, total monitoring with cumulative weight management, time setting and function setting with a weight setting.

U.S. Pat. No. 7,735,761 (“'761 patent”), contents of which are herein incorporated by reference in its entirety, discloses a food waste disposal apparatus which decomposes food waste using microbes. The '761 patent also discloses a control panel installed on the food waste disposal apparatus to operate the apparatus under auto working mode or manual working mode according to a user's instructions. The control panel controls the operation of the agitator, water supply into a mixing basin, washing, microbes supply, and discharge valve. Under the manual working mode, a user can operate each function of the apparatus whereas under the auto working mode, the apparatus works as programmed.

However, the operations of agitator, water supply, washing, drug supply and discharging are independent to each other. Each unit repeatedly works for a preset time and stops for another preset time. The preset times for each function unit are programmed based on rough experience. Therefore, the efficiency of the apparatus is limited and its operation is not optimal. Due to inefficiency, the total operation time is relatively long, and water, electricity and drug are unnecessarily wasted.

Moreover the inventor updated the food waste disposal apparatus by adding more functions such as weight management and alarming system for overloading in US utility patent application Ser. No. 13/304,516.

The unit represented in the application Ser. No. 13/304,516 is also able to communicate the computer through the internet connection by using the Ethernet card embedded in the touch panel unit. So, the user and the technical assistant can monitor the unit's operational status and runs the machine remotely. The update of Ethernet networking option considerably saves the maintenance and repair time when the unit is stopped due to the user's simple mistake.

Accordingly, a need for a new control device for a food waste disposal apparatus to improve efficiency and optimal operation of the apparatus has been present for a long time considering the expansive demands in the daily operation. This invention is directed to solve these problems and satisfy the long felt need.

### SUMMARY OF THE INVENTION

The present invention contrives to solve the disadvantages of the prior art. An object of the invention is to provide a control device for a food waste disposal apparatus to achieve an efficient and optimal operation of the apparatus.

Another object of the invention is to provide a food waste disposal apparatus, which can be operated in response to instructions fed from the control device, the control device comprising input means; a memory device; means for processing instructions to control the apparatus; means for auto working mode; means for manual working mode; means for time setting; and means for function setting, comprising a weight setting.

Another object of the invention is to provide a control device for a food waste disposal apparatus, further comprising means for total monitoring, cumulative weight management, and part replacement cycle management. Total monitoring is to check the operation status of the apparatus' each function unit. Cumulative weight management stores data and information of each operation and these data and information are analyzed to produce optimal operation mode of the apparatus and prevent the user from overloading the food waste disposal system. Furthermore, the part replacement cycle management makes the user inspect major parts of the apparatus when their life is almost over.

Still another object of the invention is to provide a food waste disposal apparatus, which comprises the remote control unit by internet networking connection, so the user can monitor and operate the unit remotely through the Ethernet system embedded in the touch control panel.

The advantages of the present invention are: (1) the control device for a food waste disposal apparatus achieved efficient and optimal operation of the apparatus; (2) the operation data are automatically stored to be analyzed to improve efficiency of the operation of the apparatus; (3) part replacement cycle management improves efficiency in maintenance of the apparatus; and (4) the controlling method for operating a food waste disposal apparatus uses various sensors to integrate the whole operation of the apparatus, resulting in efficient and optimized operation of the apparatus.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings.

FIG. 1 is a side view of the apparatus operated by the control device of the present invention.

FIG. 2 is a schematic diagram depicting the operation of the apparatus operated by the control device of the present invention.

FIG. 3 is a touch screen displaying main menu of the control device of the present invention.

FIG. 4 is a touch screen displaying auto working mode of the control device of the present invention.

FIG. 5 is a touch screen displaying manual working mode of the control device of the present invention

FIG. 6 is a touch screen displaying time setting of the control device of the present invention.

FIG. 7 is a touch screen displaying function setting 1 of the control device of the present invention.

FIG. 8 is a touch screen displaying another function setting 2 of the control device of the present invention.

FIG. 9 is a touch screen displaying still another function setting 3 of the control device of the present invention.

FIG. 10 is a touch screen displaying total monitoring of the control device of the present invention.

FIG. 11 is a touch screen displaying the door open and close history log of the present invention.

FIG. 12 is a touch screen displaying part's replacement cycle of the control device of the present invention.

FIG. 13 is a touch screen displaying Ethernet networking setup of the control device of the present invention.

### DETAILED DESCRIPTION EMBODIMENTS OF THE INVENTION

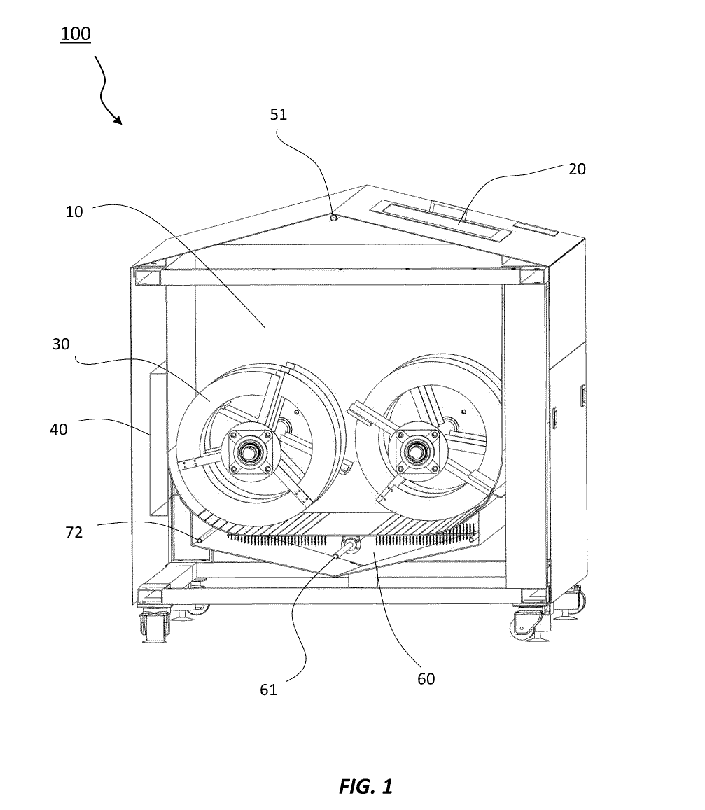

FIG. 1 shows a side view of the apparatus 100, comprising a mixing basin 10, a door 20 to input food waste into the mixing basin 10, an agitator 30 installed in the mixing basin 10 to mix the food waste, a hot water tank 40 provided with a heater to heat the mixing basin, a microbe tank, a drain water tank 60, and a washing device. The hot water tank 40 is equipped with heaters to heat up water and the hot water in the tank 40 is used to conductively heat the mixing basin 10. Heating and maintaining the temperature of the mixing basin 10 is very important for the operation of the apparatus 100 because the activity of microbes is known to be the best at a certain temperature level. Micro-organisms are the most active at 37-42° C. If the temperature of the mixing basin 10 is higher or lower than this range, micro-organism activity drops quickly, resulting in low performance of the apparatus. Once food waste is placed into the mixing basin 10 and the temperature of the mixing basin reaches a predetermined level, preferably at around 15° C., the agitator 30 begins rotating to mix the food waste. Then, microbes in the microbe tank are periodically sprayed onto the food waste through the spray nozzles 51. The microbes decompose protein, fat, sugar, fatty acids, and cellulose in the food waste. By the decomposition of food waste by microbes, leachate is generated and collected in the drain water tank 60, and then, it is periodically discharged out of the apparatus through a drain pipe 61.

The apparatus 100 further comprises a cleansing device to clean the mixing basin and the drain water tank using high-pressure water and air. After complete decomposition of the food waste, to clean the mixing basin 10 and the drain water tank 60, hot water in the hot water tank 40 is sprayed at a high speed into the mixing basin 10 through the spray nozzles 51 and into the drain water tank 60 through the drain tank spray nozzles 72. Then, high pressurized air is supplied into the mixing basin 10 to blow down water therein down into the drain water tank 60 to be discharged out of the apparatus 100.

The food waste disposal system unit 100 under this invention is the same as the food waste disposal system posted U.S. Pat. No. 7,735,761, U.S. Pat. No. 7,762,713 and U.S. application Ser. No. 13/304,516 by the same inventor of this application, so the detailed description thereof will be left out.

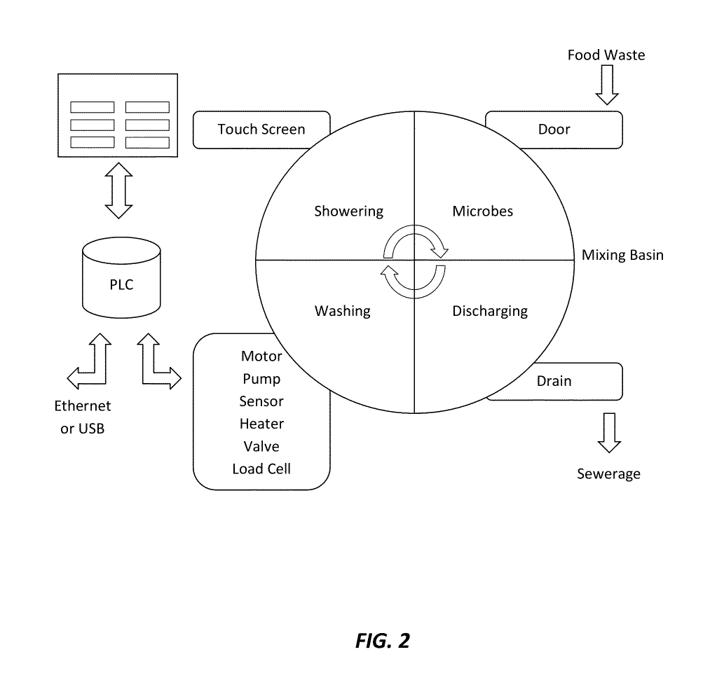

FIG. 2 is a block diagram depicting the operation of the apparatus operated by the control device of the present invention. Above-described operation of the apparatus 100 is performed by a control device comprising an input means, a processor and a memory device. Preferably, a touch screen is used as an input means and a display device.

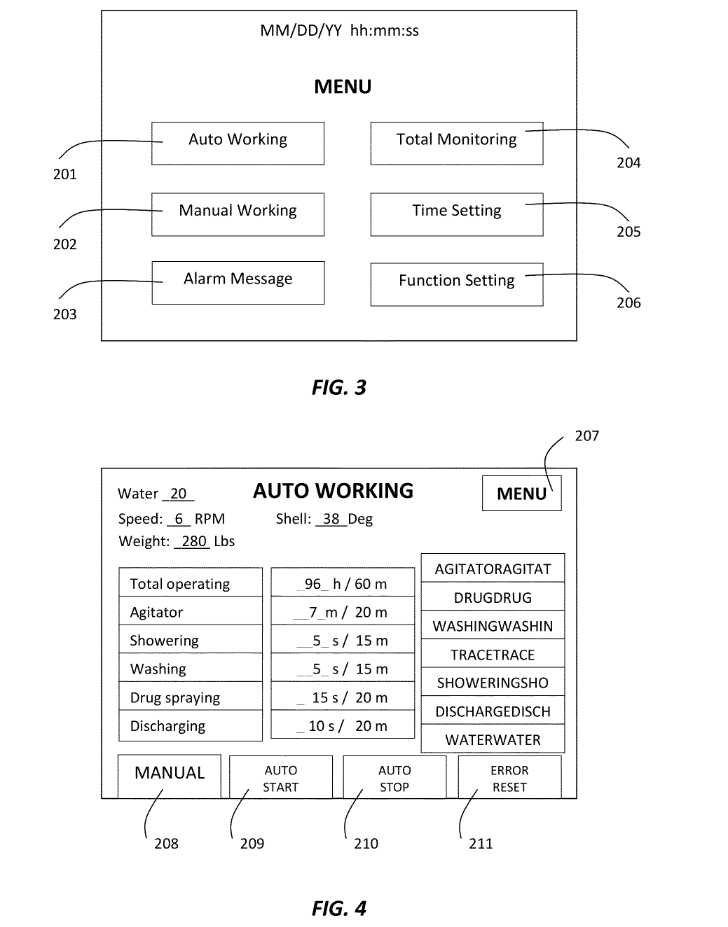

FIG. 3 shows a touch screen displaying main menu, which comprises auto working button 201, manual working button 202, alarm message button 203, total monitoring button 204, time setting button 205, and function setting button 206. The control device for use in controlling a food waste disposal apparatus, which can be operated in response to instructions fed from the control device, comprises: input means being operable by a user for inputting an instruction or information; a memory device for storing data relating to the operation of the apparatus; means for processing instructions to control the apparatus.

Under the auto working mode, the apparatus 100 is operated as programmed by the time setting mode. FIG. 4 shows the auto working mode where for instance, the agitator rotates 7 minutes out of 20 minutes' operation. A user may change the time setting and the function setting for the auto working mode conditions by clicking time setting button 205 in main menu screen. In the auto working mode, the user can monitor the agitator's speed and the temperature in the mixing basin as well as the amount of the food waste inside the unit. The user initiates the auto working mode by clicking the auto start button 209 at the bottom of the screen and stop auto mode by auto stop button 210. If the unit automatically stops the operation due to the error or overloading, the cause of the error should be eliminated and push error reset button 211 to clear the memory inside the PLC. Manual button 208 at the bottom of the screen switches the screen to manual working mode. The user can go back to main menu by clicking menu button 207.

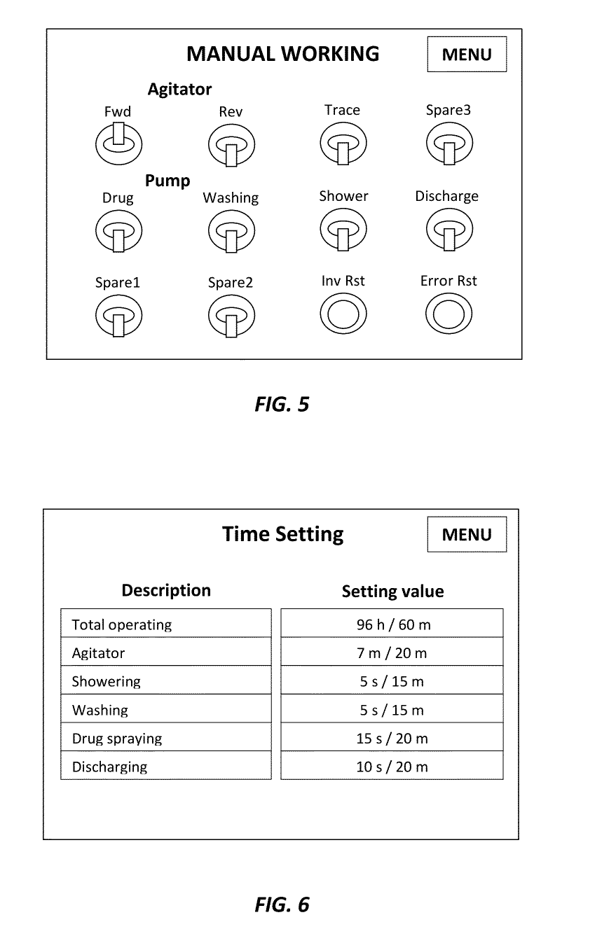

Under the manual working mode through the manual working button 202 in the main menu or manual button 208 in auto working mode, the apparatus 100 is operated pursuant to the instructions by the user. FIG. 5 shows toggle switches for each part of the apparatus 100. By toggling on and off, a user can turn each part of the apparatus 100 on and off. For example, turning Agitator toggle switch upward in the FIG. 5 turns on the agitator rotation. The switch operates the part independently and this mode is conveniently used for the user to check out the part whether it is running or out of order.

A user may change the time setting for the auto-working mode using the time setting as in FIG. 6 through the time setting button 205. The input values of the time setting change depending on the kind of food waste or environment where the apparatus 100 is installed. The time setting sets the part's running time by inputting two numbers. The first number is the duration of part's running time and the second number is one cycle time. For instance, the showering shows 5 s/15 m. This means that the showering operation is performed such that the showering unit sprays the water for 5 seconds every 15 minutes. The symbol used in the setting is conventionally abbreviated, h represents hour, m minute, and s second in time dimension. The user may change the operation time and cycle time of each part, but most likely, the optimal condition is set by the factory so as to minimize any unit operation by the user. The value in FIG. 6 is one of the examples that can be widely used in various operating condition.

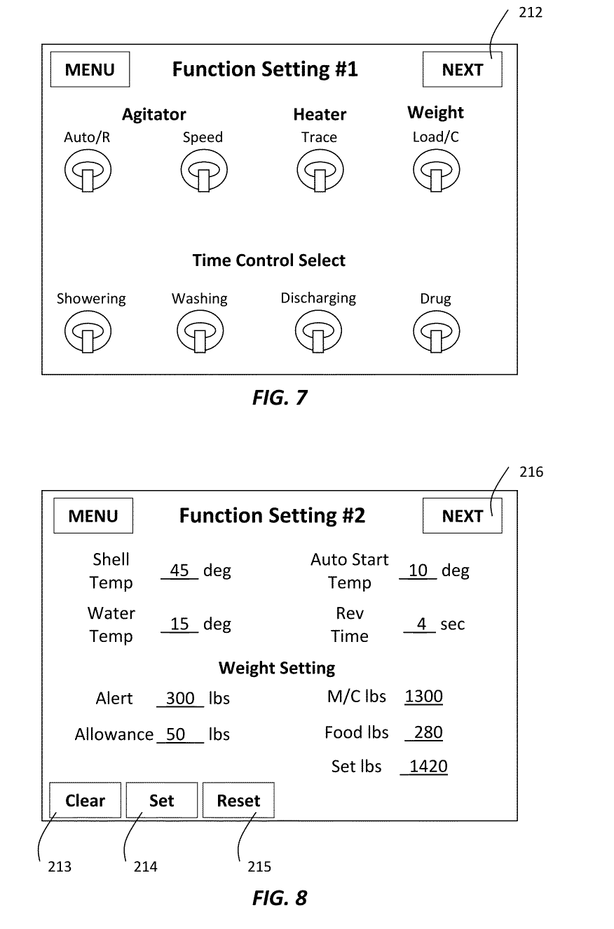

The function setting 1 through function setting button 206 allows the user to set conditions for the auto working mode. By turning on or off switches in FIG. 7, a user can change what function units are used under the auto working mode. A user can change the agitator speed as well by turning on the switch to be 8 rpm (revolution per minute), or turn off to be 6 rpm that is default setting. The main reason to function setting 1 is that the user can take some options out from the operation. For example, in the place that operating environment is in high temperature, there is no need to use the heater, so the user can switch off the heating function in this mode.

FIG. 8 through clicking next button 212 shows the function setting 2. Shell Temperature is set to 45 degree Celsius and if the temperature of the mixing basin goes over this setting, the unit automatically stops the operation to protect the microbes from overheat. Auto Start Temperature is set to 10 degree Celsius. To decompose the organic waste, the mixing basin should be maintained between 10 and 45 degree Celsius. The heater in the unit will be turned on and the unit does not start auto working mode until the temperature of the mixing basin to be Auto Start Temperature setting. Water temperature setting is inlet water's minimum temperature and if the water temperature is below this setting, the unit stops the operation. Rewinding Time sets the duration of rewinding motion of the agitator. If the hard material is stuck between the agitator and the mixing basin, the load to the motor goes up and the agitator rotates reversely to unlock the hard material. Rewinding Time is normally set to 4 second.

Another setting in the function setting 2 is the weight setting. The unit controls the total amount of food waste input based on the following setting values. Alert is the nominal amount of the food waste inside the mixing basin. If the user put more food waste than Alert value, the touch screen sends the warning signal to stop putting the waste into the unit. Preferably, the maximum value is 500 lbs. for 1500 lbs. apparatus, 400 lbs. for 1200 lbs. apparatus, 300 lbs. for 800 lbs. apparatus, 200 lbs. for 400 lbs. apparatus, and 100 lbs. for 200 lbs. apparatus.

Allowance is the buffering weight. When the user opens and closes the door, there is some impact force upon the unit that affects the food waste weight measurement. Allowance is set to make a room for this kind of sudden change that is not related to the food waste weight. Typically, Allowance is set to 50 lbs. When the user put more food than the sum of Alert and Allowance value, the unit stops the operation and does not restart until the user takes out the food waste below the Alert weight. This will prevent the unit breakage from the overloading of food waste into the unit, educating the user to use the unit properly by itself. To measure the accurate weight of food waste, the unit should recognize the several basic weights.

M/C lbs. represent the weight of the unit itself and this value will be the base weight for calculation. Set lbs. is the weight of the unit when it's in operation. When the unit is running, there are many factors that make the total weight of the unit to be changed. When showering function working, the water is sprayed into the mixing basin and the weight of the unit is increased even there is no change in terms of the food waste weight. So, Set lbs. represent the total weight of the unit including waters and microbes except the food. This value can be set by clicking Set button 214 at the bottom of the screen. It is usually done when the unit is installed at the site for the first time and should be memorized in the system.

Food lbs. shows only the food waste input in the unit. This value is calculated simply by current weight by the load cell is subtracted by the Set lbs. Any setting in this function setting 2 can be cleared by clicking Clear button 213 or Reset button 215 and changed by putting the desired numbers directly into each underscore session.

When next button 216 is clicked, the screen goes to Function setting 3. In this mode, the user can put the Set lbs. value into the PLC memory so that the value should be memorized and recurred in case the unit is moved from one place to another or restart after rebuilt. To save the Set lbs., the unit's dimension toggle switch should be activated first. The food waste decomposition system carries five different models and the user should select an appropriate model according to its capacity. Then the box at the bottom is activated and allowed to input the numbers by clicking the write button.

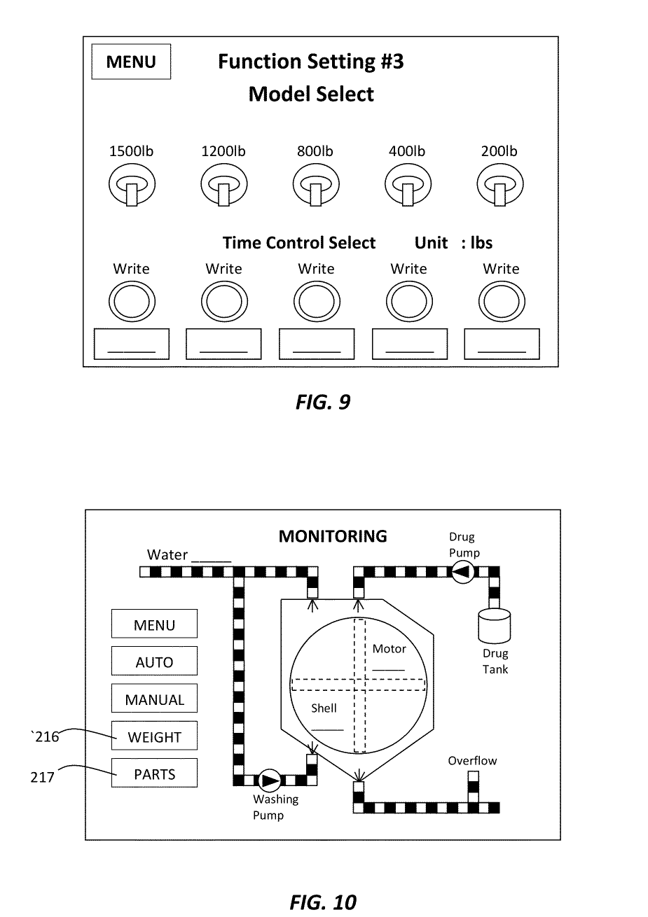

FIG. 10 shows the total monitoring through total monitoring button 204 in the main menu, which allows the user to check operation status of the apparatus. The total monitoring also includes cumulative weight management and part replacement cycle management. The weight management stores information of time and weight of the food waste processed by the apparatus. The user can go to the weight management screen by clicking Weight button 216.

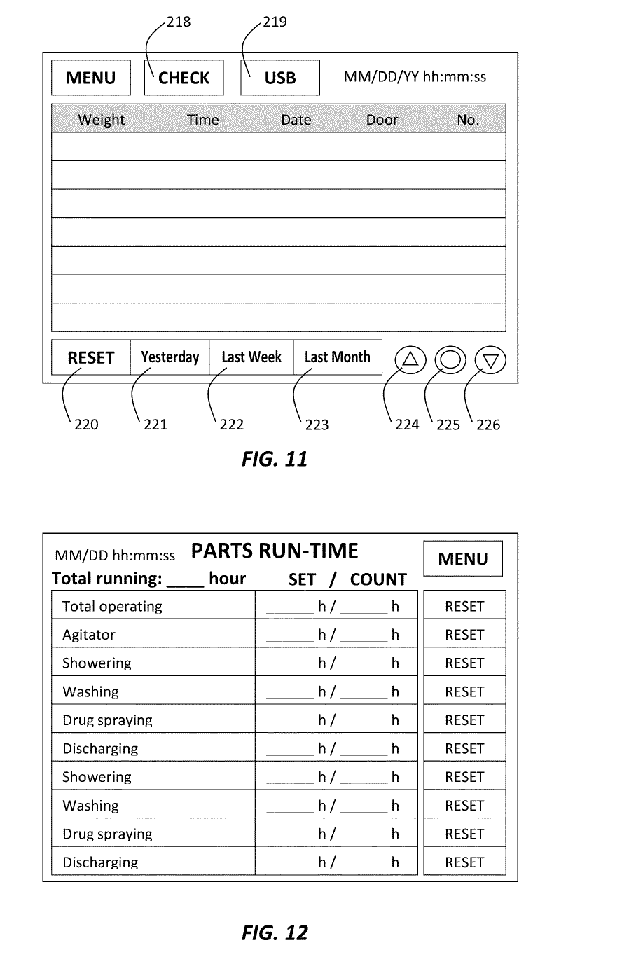

FIG. 11 shows the typical screen of the weight management. The user can see all the history of the input of food waste in this mode. It records the date and time and door open-close status and amount of food waste input. This data shows the total amount of food waste processed by the unit in daily, weekly and monthly basis. Yesterday button 221 shows the daily amount of food waste, Last week button 222 to weekly, and Last month 223 to monthly. If the user wants to see current weight status, the value is recorded by clicking Check button 218. This data also is possible to be saved in external devices such as USB by clicking USB button 219. When the record data is more than the screen lines, the user can scroll up and down using the arrow buttons 224, 225, 226.

Another feature in total monitoring screen is the part management which can be accessed through Parts button 217. Part replacement cycle management stores expected length of life for each part of the unit and sends alarm to the user if the time is almost up for a particular part of the unit. The user should be aware of the part's life cycle and call the maintenance support team so that the unit is always up to the best operation condition. All the parts have operation length in hour basis and the count shows the actual usage time.

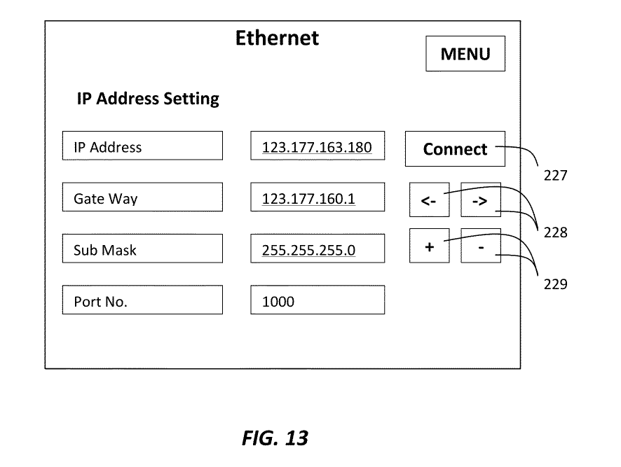

FIG. 13 shows the touch panel screen for Ethernet card setting. This mode is shown only when the user purchase the remote control options on the unit. So, this mode can be accessed through the external button on the touch panel, which is not displayed on the screen. When the button is clicked, the user can see the network setting screen, FIG. 13. The user can set up the IP address, gate way, and subnet mask, by using the arrow 228 and add and subtract button 229. This information required in the network system is same as other network setting. Once all the information is entered, the networking system starts working when clicking Connect button 227 and now the user can see all the information through the internet and remotely control the unit without any restriction.

The control device further includes means for alarm message which sends alarm for specific function of the apparatus. Preferably, access to means for manual working mode, means for time setting, and means for function setting is password protected so that only an authorized user can have access thereto. Ordinary users are advised to use only auto working mode, total monitoring and alarm message. The apparatus may further comprise a water level sensor so that the drain water tank discharges water for a predetermined time if water level measured by the water level sensor reaches a predetermined water level. Alternatively, the drain water tank discharges water if water level reaches the predetermined water level or a predetermined time passes, whichever is earlier. All of the error or alarm message can be shown through the alarm message button 203.

The function setting may further comprise a control switch wherein if the control switch is on, the operation of the auto working mode is initialized whenever the door opens and closes whereas if the control switch is off, the operation of the auto working mode is not initialized irrespective of the door being open or closed. In the off mode of the control switch, water consumption can be saved. Besides, the function setting may additionally comprise a drug switch wherein if the drug switch is on, sanitizing agent are supplied according to the time setting irrespective of the door being open or closed whereas if the drug switch is off, sanitizing agent can be supplied only when the door is open. The on mode of drug switch is preferably used when the food waste is the type of one exuding a lot of bad odor. Preferably, the input means is a touch panel or a touch sensitive panel for generating a plurality of output signals for the operation of the apparatus where the touch sensitive panel is contacted with a finger. Furthermore, the control device may be a PLC.

For efficient and optimal operation of the apparatus 100, the controlling method for operating a food waste disposal apparatus, which comprises a mixing basin, an agitator installed in the mixing basin to mix the food waste, a microbe tank, ′ and a drain water tank, comprises the steps of heating and maintaining temperature of the mixing basin to a predetermined temperature range; regularly mixing the food waste using the agitator; measuring water level of the drain water tank; calculating water level increasing speed in the drain water tank; supplying microbes into the mixing basin if the water level increasing speed drops below a predetermined level or a predetermined time is up, whichever is earlier. Here, the apparatus 100 may additionally comprise a door to input food waste into the mixing basin, a hot water tank provided with a heater to heat the mixing basin, and a washing device.

The apparatus 100 may further comprise a door open/close sensor to detect whether the door is open or closed and a weight scale to measure the weight of the mixing basin, and the controlling method may further comprise determining weight of the food waste inputted into the mixing basin by comparing weights of the mixing basin before and after inputting the food waste.

The apparatus 100 may further comprise a thermometer, a water level sensor, a hygrometer to measure the humidity inside the mixing basin and flow meters to measure the amount of water and microbes supplied to the mixing basin. Using these sensors, a certain amount of water can be supplied to maintain a certain level of humidity insider the mixing basin and a certain amount of microbes can be supplied to maintain the speed of decomposition of food waste. The controlling method may further comprising regularly supplying water into the mixing basin, and washing the mixing basin and the drain water tank using a high-pressure water and air after complete decomposition of the food waste. While the invention has been shown and described with reference to different embodiments thereof, it will be appreciated by those skilled in the art that variations in form, detail, compositions and operation may be made without departing from the spirit and scope of the invention as defined by the accompanying claims.

1. A control device for use in controlling a food waste disposal apparatus comprising;
a touch screen located on a top surface of the control device for receiving instruction and data from a user;
a memory device attached inside the control device for storing data inputs related to the operation of the food waste disposal apparatus;
wherein, a main menu displayed on the touch screen of the control device is comprised of a plurality of button images shown to an auto working mode, a total monitoring mode, a manual working mode, a time setting mode, an alarm message mode and a function setting mode;
wherein, under the auto working mode, a user operates the food waste disposal apparatus automatically;
wherein, under the manual working mode, the apparatus is operated pursuant to the instructions by the user;
wherein, the user is able to check out the alarm signal for a specific event of the food waste disposal apparatus;
wherein, the total monitoring allows the user to check current operation status of the food waste disposal apparatus;
wherein, the time setting allows the user to set time intervals for each function of the food waste disposal apparatus;
wherein, the function setting allows the user to set conditions for the operation of the food waste disposal apparatus;
wherein, the total monitoring menu is comprised of a plurality of button images shown to a cumulative weight setting, a part replacement cycle setting, and a remote control mode;
wherein, the weight setting stores information of time and weight of the food waste processed by the apparatus;
wherein, the weight setting allows the user to set weight of empty mixing basin measured by a load cell so that weight of the food waste inputted into the mixing basin can be determined by weight of the mixing basin with the food waste measured by load cell subtracted by the weight of the mixing basin;
wherein, the weight setting allows the user to set maximum allowable weight of food waste for the apparatus;
wherein, a buffering weight is set to make a room for sudden weight change that is not related to the food waste weight;
wherein, the part replacement cycle management stores expected length of life for each part of the food waste disposal apparatus and sends alarm to the user if the time is almost up for a particular part of the food waste disposal apparatus; and
wherein, the remote control mode is set by the Ethernet card embedded in the control device and the food waste disposal apparatus can be monitored and operated remotely through an internet.

2. (canceled)

3. (canceled)

4. The control device of claim 1, wherein the manual working mode, the time setting mode, and the function setting mode are password protected so that only an authorized user can have access thereto.

5-6. (canceled)

7. The control device of claim 1, wherein the function setting mode is further comprised of a plurality of control switch images;
wherein if the control switch is on, the operation of the auto working mode is initialized regardless of the status of a door of the food waste disposal apparatus, whereas if the control switch is off, the operation of the auto working mode is not initialized irrespective of the door being open or closed; and
wherein, the auto working mode is run by the values set in the time setting mode.

8. (canceled)

9-11. (canceled)

12-18. (canceled)
